(19) United States
(12) Patent Application Publication
(10) Pub. No.: US 2024/0099848 A1
(43) Pub. Date: Mar. 28, 2024

(54) Implantable Medical Device for Modulation of Cerebrospinal Fluid and Glymphatic Flow

(71) Applicant: Victor M. Haughton, Oconomowoc, WI (US)

(72) Inventor: Victor M. Haughton, Oconomowoc, WI (US)

(21) Appl. No.: 18/466,189

(22) Filed: Sep. 13, 2023

Related U.S. Application Data
(60) Provisional application No. 63/376,825, filed on Sep. 23, 2022.

(51) Int. Cl. A61F 2/28; A61H 23/02

(52) U.S. Cl. CPC A61F 2/2875; A61H 23/0245; A61B 1/04

(57) ABSTRACT

An implantable medical device that has a source of renewable power and has a mechanical structure to expand or collapse at a selected rate and volume to modify intracranial pressure and cerebral fluid circulation. The implantable medical device may be programmed to increase intracranial pressure variably to a prescribed amount in the range of several pascals cyclically and at a prescribed frequency, notably at 0.05 Hz, to produces cyclic intracranial pressure variations. Typically those amplitudes and frequencies drive the glymphatic flow in the brain during deep sleep. This allows the implantable medical device to provide pressure oscillations at prescribed time, for example during NREM sleep, that boost glymphatic CSF circulation in the brain, and which remove toxic and metabolic waste from the brain. The glymphatic circulation may protect patients from neurodegenerative diseases, most notably Alzheimer's Disease. The implantable medical device is intended to improve the rinsing of toxins and metabolites from the brain in patients who lack normal glymphatic circulation, such as patients with Alzheimer's Disease. It also provides the means to monitor intracranial pressure and cranial fluid volume fluctuations. It may be used in patients and in animal models of neurodegenerative disease for diagnosis, monitoring CSF mechanics, or for increasing the rinsing of metabolites and toxic substances impairing neurologic function and for preventing or delaying neurodegenerative disease and maintaining neurologic functions in patients with neurodegenerative disease.

### RELATED APPLICATIONS

This application claims the benefit of U.S. Provisional Patent Application Ser. No. 63/376,825, filed on Sep. 23, 2022, which is incorporated by reference in its entirety.

This application is related to U.S. Provisional Patent Application Ser. No. 62/173,820, filed on Jun. 10, 2015, which is incorporated by reference in its entirety.

This application is related to U.S. patent application Ser. No. 15/176,705, filed on Jun. 8, 2016, now U.S. Pat. No. 10,207,089 B2, which is incorporated by reference in its entirety.

### FIELD OF THE DISCLOSURE

The present invention relates to the field of neurosurgical devices and procedures, and in particular, implantable medical devices and procedures for alleviating neurological disorders associated with abnormal cerebrospinal fluid (CSF) flow including Alzheimer's Disease (AD) and Parkinson's Disease. The invention has the capability to improve fluid flow in the glymphatic circulations thereby hastening the clearance of toxins and metabolites from the brain.

### BACKGROUND

Since the normal human skull is rigid, transient volume variations in intracranial structures (such as cerebral vasculature and brain) cause the intracranial cerebrospinal fluid (CSF) to flow to or from the cranial vault and the spinal canal. (Mokri 2001). As an example, the arterial pulse wave in the cranial vault, drives CSF flows cyclically through the foramen magnum that connects the cranial vault and the spinal canal.

The implantable medical device described in this application has the capability to alter the pressure wave in the cranial vault. It may be programmed to produce a pressure wave at a selected frequency and amplitude to produce the desired expansion of the intracranial fluid volume. This will drive glymphatic fluid and thereby improve the rinsing of toxins and metabolites from the brain. This provides a new method of treatment for patients with neurodegenerative disorders.

The implantable medical device may employ the same type of implantable MRI-compatible chamber that is used in U.S. Pat. No. 10,207,089 B2, armed with a mechanism that variably and reversibly removes fluid from the subarachnoid space. The implantable medical device may include instrumentation that monitors the function of the device, such as monitors of intracranial pressure fluctuations and determines the need for a changing the volume of the CSF flow diversion.

This application describes such added functionalities not present in U.S. Pat. No. 10,207,089 B2. The mechanism to move fluid may be mechanical, driven by a motor, or a material that changes shape or size due to a thermal or electric effect (including a piezoelectric effect).

### SUMMARY

This application describes an implantable medical device for placement within a small craniotomy in the calvarium in a region of the head that is easily accessible. The implantable medical device has a chamber, easily accessed before or after implantation, in which mechanisms may be placed for the purpose of monitoring and modifying intracranial CSF dynamics. The implantable medical device has a source of renewable power and has a mechanical structure to expand or collapse at a selected rate and volume to modify intracranial pressure and intracranial fluid circulation.

The implantable medical device provides pressure oscillations at prescribed times, for example during NREM sleep, that boost glymphatic CSF circulation in the brain and remove toxic and metabolic waste from the brain. The glymphatic circulation hypothetically protects patients from neurodegenerative diseases, most notably AD and Parkinson's Disease. The implantable medical device is intended to treat neurodegenerative diseases by improving the rinsing of toxins and metabolites from the brain in patients who lack normal glymphatic circulation. It also provides transducers to monitor intracranial pressure and fluid volume fluctuations. It may be used in patients and in animal models of neurodegenerative disease for diagnosis, monitoring CSF mechanics and increasing the rinsing of metabolites and toxic substances that degrade neurologic function. It may prevent or delay neurodegenerative disease and maintain neurologic functions in patients with neurodegenerative disease. The device may also be used to house other devices to provide electrical stimulation, tracer injection, chemotherapy into the cranial vault.

The implantable medical device may be programmed to increase intracranial pressure to a prescribed amount, notably in the range of several pascals and cyclically at a prescribed frequency, notably at 0.05 Hz to produces cyclic intracranial pressure variations typical of those amplitudes and frequencies that drive the glymphatic flow in the brain. One of the mechanisms that fits into the chamber may produce pressure waves in the cranial vault designed to promote glymphatic and/or CSF flow. This may aid research in glymphatic circulation in animal models and in treatment of patients.

### BRIEF DESCRIPTION OF THE FIGURES

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a top view of a fully assembled implantable medical device in accordance with some embodiments.

FIG. 2 is a bottom view of a fully assembled implantable medical device in an accordance with some embodiments.

FIG. 3 is an exploded top view of an implantable medical device in accordance with some embodiments.

FIG. 4 is an exploded bottom view of an implantable medical device in accordance with some embodiments.

FIG. 5 is a front sectional view of an implantable medical device in accordance with some embodiments.

FIG. 6 is a front sectional view of an implantable medical device with additional features in accordance with some embodiments.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

### I. Introduction

The implantable medical device contains a structure that effectively contracts when the CSF pressure rises and expands when CSF pressure decreases. The implantable medical device may include a chamber with the property of changing in volume as intracranial pressure changes. The implantable medical device may be a passive system comprised of a flexible membrane or flexible membranes that covers the chamber, providing a standard compliance greater than normal cranial compliance (change in volume for change in pressure). Or the implantable medical device may have an experimentally determined compliance charged with a known (or experimentally determined) value that serves to provide the compliance required to address the adverse CSF pressures associated with the blockage of the normal CSF channels or abnormal compliance in the cranial vault. Alternatively, the implantable medical device may consist of elements actively controlled by either a prescribed program or by dynamic or static measurements of the existing CSF pressure values and actively adjusted (at or near real time) or adjusted by a nominal control value to alleviate adverse CSF pressure values. Alternatively, the implantable medical device may use materials that change shape with the application of heat, electric current, magnetic forces, or microfluidics.

Further, the implantable medical device may be configured to be attached to a spinal column in such a way as to modify the spinal column's effective compliance. The implantable medical device may further include a monitor that allows external non-invasive monitoring of the device's functioning. The implantable medical device may further comprise a controller that permits selectable increase or decrease of the device's compliance in either near real time (at a fixed relationship) or in real time.

### II. Nomenclature

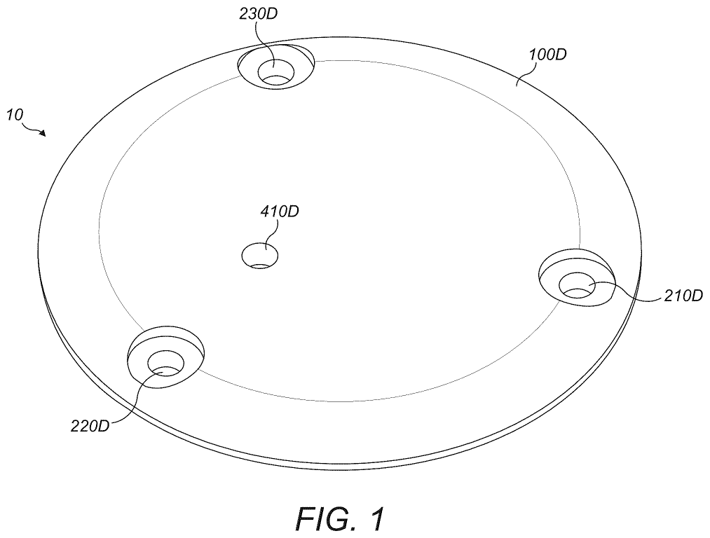

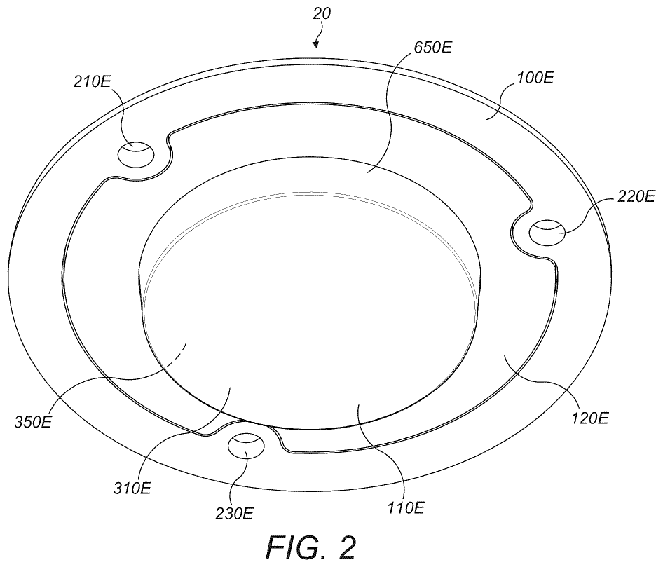

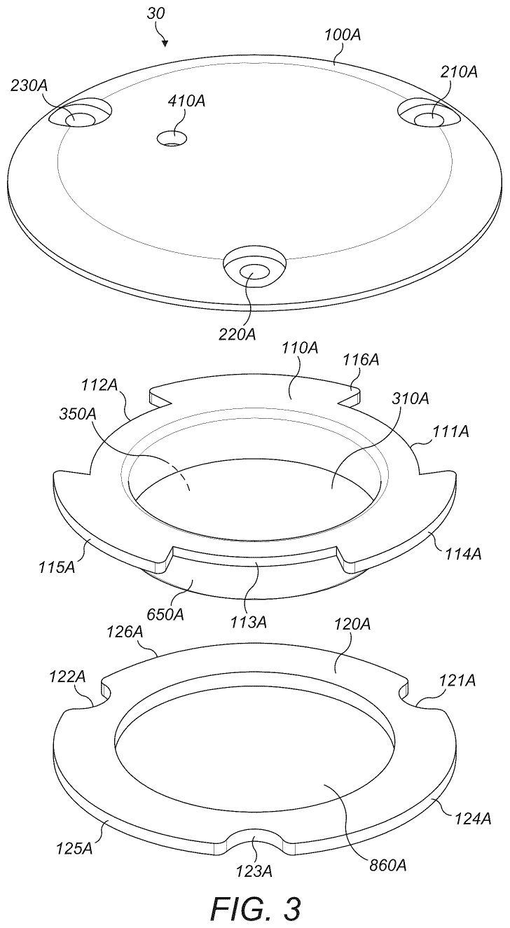

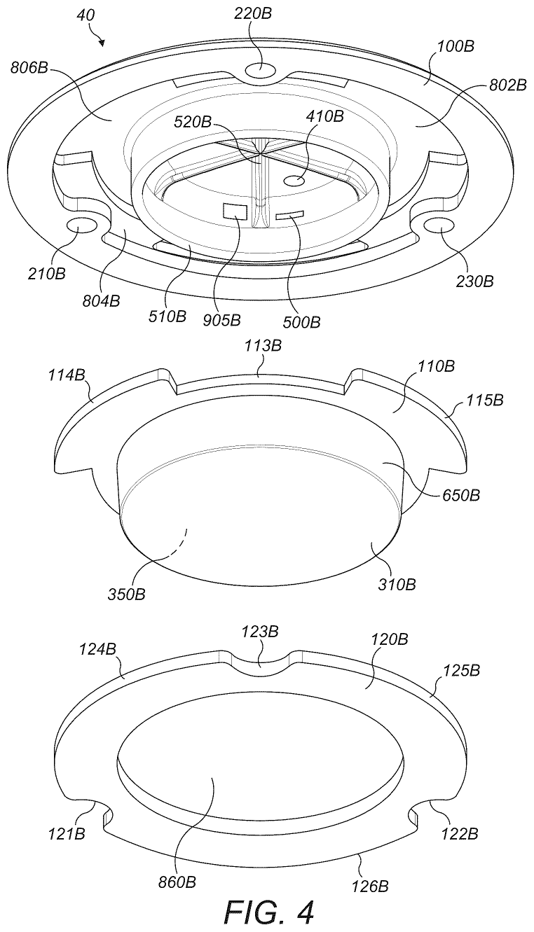

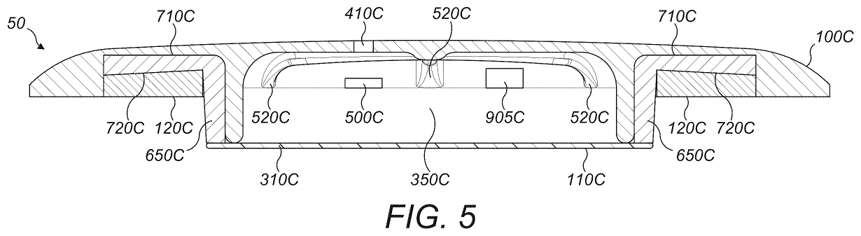

In these foregoing descriptions, each subpart of the implantable medical device shown in FIGS. 1-5 has the same numerical reference and may optionally also be referenced with a letter suffix that varies depending on which figure is being discussed. For example, the cover level 100 is shown from various angles in FIG. 1 (assembled top view 100D), FIG. 2 (assembled bottom view 100E), FIG. 3 (exploded top view 100A), FIG. 4 (exploded bottom view 100B) and FIG. 5 (section view) 100C). Thus, all of these views of the cover level 100A, 100B, 100C, 100D, 100E represent the same cover level 100.

### III. Review of the Drawings

Turning to FIG. 1, shown is a top view 10 of a fully assembled implantable medical device. Shown is a top portion of a cover level 100D which includes the top portion of a multitude of holes 210D, 220D, 230D. The plurality of holes 210D, 220D, 230D are designed to be secured to the cranium via securing mechanisms such as medical screws (not shown). Also shown is the top portion of a nib 410D that is drilled all the way through the cover level 100D so as to allow limited access to levels below the cover level 100D.

Turning to FIG. 2, shown is a bottom view 20 of a fully assembled implantable medical device. Shown is the bottom portion of a cover level 100E which includes the bottom portion of a multitude of holes 210E, 220E, 230E. Also shown is the bottom portion of a membrane level 110E and the bottom of the retaining level 120E. This level consists of the bottom portion of the flexible membrane 310E that incorporates a chamber 350E above. The membrane level incorporates a side portion 650E that is tapered so as to provide the proper interference fit to a cranium of a patient.

Turning to FIG. 3, shown is an exploded top view 30 showing the three levels of the implantable medical device: the cover level 100A, the membrane level 110A and the retaining level 120A.

The cover level 100A shown includes the top portion of a plurality of holes 210A, 220A, 230A. Also shown is the top portion of a nib 410A that is drilled all the way through the cover level 100A so as to allow limited access to levels below the cover level 100A.

The membrane level 110A incorporates a flexible membrane 310A which forms the bottom boundary of the chamber 350A (the top boundary being the cover level 100A). The membrane level 110A incorporates a side portion 650A that is tapered so as to provide the proper interference fit to a cranium of a patient. The top portion of the membrane level incorporates a plurality of sawtooth members 114A, 115A, 116A interspersed with a plurality of large cutouts 111A, 112A, 113A.

The retaining level 120A incorporates a plurality of retaining members 124A, 125A, 126A interspersed with small cutouts 121A, 122A, 123A. The retaining level 120A is designed to secure the membrane level 110A to the cover level 100A so that the membrane level 120A is sandwiched in between. When so assembled, the small cutouts 121A, 122A, 123A allow access for the securing mechanisms (not shown) to pass through the plurality of holes 210A, 220A, 230A and into the cranium of the patient. Further the side portion 650A and the flexible membrane 310A pass through the hole 860A when so assembled.

Turning to FIG. 4, shown is an exploded bottom view 40 showing the three levels of the implantable medical device: the cover level 100B, the membrane level 110B and the retaining level 120B. The cover level 100B shown includes the bottom portion of a plurality of holes 210B, 220B, 230B. Also shown is the bottom portion of a nib 410B that is drilled all the way through the cover level 100B. This view also shows the underside of the cover level 100B, showing a plurality of struts 520B providing structural support for the implantable medical device, all further secured by a ring 510B. Also shown is a stopper 500B attached to the struts 520B that is situated below the nib 410B. Also shown are a plurality of tooth holders 802B, 804B, 806B that serve as a receptacle for portions of the membrane level 110B as discussed below. Also shown is a monitoring/control mechanism 905B that may be attached to the struts 520B and allow for the monitoring and/or control of the operation of the implantable medical device.

The membrane level 110B incorporates a flexible membrane 310B which forms the bottom boundary of the chamber 350B (the top boundary being the cover level 100B). The membrane level 110B incorporates a side portion 650B that is tapered so as to provide the proper interference fit to a cranium of a patient. The top portion of the membrane level incorporates a plurality of sawtooth members 114B, 115B, 116B (116B is not shown) interspersed with a plurality of large cutouts 11B, 112B, 113B (111B, 112B are not shown). The sawtooth members 114B, 115B, 116B are designed to be bonded with the tooth holders 802B, 804B, 806B, thus securing the cover level 100B to the membrane level 110B

The retaining level 120B is ring-shaped with a hole 860B that incorporates a plurality of retaining members 124B, 125B, 126B interspersed with small cutouts 121B, 122B, 123B. The retaining level 120B is designed to be bonded with the cover level 100B with the membrane level sandwiched in between. When so assembled, the small cutouts 121B, 122B, 123B allow access for the securing mechanisms (not shown) to pass through the plurality of holes 210B, 220B, 230B and into the cranium of the patient. Further the side portion 650B and the flexible membrane 310B pass through the hole 860B when so assembled.

Turning to FIG. 5, shown is a front section view 50 of a fully assembled implantable medical device. From this view, the interfacing of the cover level 100C, membrane level 110C and retaining level 120C among each other is readily observable. In particular, the bonded areas 710C, 720C show how the retaining level 120C is used to secure the membrane level 110C to the cover level 100C. Also shown is a cutaway of the struts 520C, the nib 410C, the stopper 500C, the chamber 350C, the flexible membrane 310C, the side portion 650C and the monitoring/control mechanism 905C.

### IV. Physical Features of the Device

The cover level 100 may be made from polyether ether ketone (PEEK) as well with a hydroxyapatite coating for bone adhesion. PEEK is a colorless organic thermoplastic polymer in the polyaryletherketone (PAEK) family. PEEK is an advanced biomaterial used in medical implants, including spinal fusion devices, reinforcing rods and other orthopedic procedures. The cover level 100 may also be comprised of plastics, titanium, or other metals that may be compatible with MRIs and transparent to ultrasound. The nib 410 may be made from a rubber-like material through which a needle can be advanced with a rigid part to hold the rubber in place and a guard to prevent the needle from entering the chamber 350 too deeply.

The membrane level 110 may also be made from PEEK, plastics, or titanium or other metals that may be compatible with MRIs and transparent to ultrasound, except that the flexible membrane 310 may be fabricated from materials such as ultra-high-molecular-weight polyethylene (UHMWPE) or the like compliant bio-compatible materials provides the compliance described herein. UHMWPE has a long history as a successful biomaterial for use in hip, knee, and spine implants. The flexible membrane 310 may also be comprised of a flexible metal alloy diaphragm.

The retaining level 120 may be made from PEEK, plastics, titanium, or other metals that may be compatible with MRIs and transparent to ultrasound.

### V. Operation of the Device

The implantable medical device is configured to reversibly increase the capacity of the cranial vault in response to a pressure change in the CSF. The implantable medical device is designed for placement in a craniotomy, or, in lay terms, a hole created surgically in the skull. An implantable medical device placed in the skull decreases CSF pressure gradients and thus reduces CSF flow through the foramen magnum, and reduces CSF velocities in the cervical spine. The implantable medical device may be placed in the craniotomy defect in place of a bone flap. Alternatively, the elastic chamber or cushion or materials in the chamber may be placed in the cranial vault. The device contains biocompatible materials where it is in contact with the human body.

The implantable medical device may be made in variable sizes and configurations typically between 11 mm and 18 mm in diameter, for example, or more particularly, 16.5 mm in diameter.

The chamber 350 may include a compressible material located therein and the volume of the chamber 350 is selectively increased or decreased in volume by the ongoing displacement of the flexible membrane 310, thereby providing the desired clinical relief by diverting the CSF flow.

The volume in the chamber 350 may be modified by intervention from the treating physicians. For example, a surgeon or physician may administer or remove gas (e.g. nitrogen or oxygen) through the nib 410 into the chamber 350 to achieve suitable pressure to increase or decrease the overall diversion of the CSF flow. The stopper 500 is situated below the nib 410 so as to prevent the needle piercing the nib 410 from going beyond the stopper 500 and puncturing or damaging the flexible membrane 310.

The chamber 350 may be filled with compressible gas such as oxygen or nitrogen (administered via the nib 410). Other possibilities for inclusion in the chamber include: 1) a compressible tissue such as foam rubber or a compressible mechanism such as a chamber fitted with springs; 2) utilizing an electric or bio-powered control element such as a pump or “Shape Memory Alloy” wherein the shape is proportional to a controlled temperature that may be altered electronically by utilizing heating or cooling systems as well as the natural surrounding temperatures.

The size of the chamber 350 may be designed to be directly proportional to the effect on CSF flow. For example, a circular chamber with a 1 cm diameter membrane that moves 3 mm between systole and diastole would reduce Foramen Magnum flow in the average adult from 2 mL to 1 mL. Such movements would modify the CSF pressure to reduce CSF flow and reduce CSF velocities in the spine and improve patient comfort and reverse the growth of syringomyelia. Putting this another way, the chamber 350 may vary in volume by an exemplary 1 mL to 2 mL during the cardiac cycle, which serves to reduce the amount of fluid displaced from the cranial vault through the Foramen Magnum by any adjustable amount, such as from 50 to 100%.

The chamber 350 may also enclose a monitoring/control mechanism 905, which may include a battery, sensors, transmitters and assorted housings. The monitoring/control mechanism 905 may include additional electronic elements designed to provide either near real time or a fixed pressure delivery protocol utilizing pressure sensors, strain gages, or the like designed to monitor and/or alter the operation of the chamber 350 and the flexible membrane 310. There may be elements that can be remotely programmed to provide the level of compliance based on the near real time measurement of the CSF pressures or on signals that encode membrane movement or intracranial volume. With this system the patient can be assured of proper CSF pressure and flow control to provide better outcomes. Well known wireless communication protocols not requiring the attachment of wires to the device may allow the surgeon to provide the necessary adjustments based on other external measurements and tests to provide a swift adjustment for the patient.

### VI. Additional Functionality of the Device

A. Introduction

The glymphatic system is the subject of ongoing research. Examples include the following.

“The glymphatic system is a recently discovered macroscopic waste clearance system that utilizes a unique system of perivascular channels, formed by astroglia cells, to promote efficient elimination of soluble proteins and metabolites from the central nervous system. Besides waste elimination, the glymphatic system may also function to help distribute non-waste compounds, such as glucose, lipids, amino acids, and neurotransmitters related to volume transmission, in the brain. Intriguingly, the glymphatic system function mainly during sleep and is largely disengaged during wakefulness. The biological need for sleep across all species may therefore reflect that the brain must enter a state of activity that enables elimination of potentially neurotoxic waste products, including β-amyloid . . . indicating that glymphatic function is suppressed in various diseases and that failure of glymphatic function in turn might contribute to pathology in neurodegenerative disorders, traumatic brain injury and stroke.” (Jessen N A et al. The Glymphatic System: A Beginner's Guide. Neurochem Res. 2015 December; 40(12): 2583-99.) (citations omitted).

“Sleep is crucial for both high-level cognitive processing and also basic maintenance and restoration of physiological function. During human non-rapid eye movement (NREM) sleep, the electroencephalogram (EEG) exhibits low-frequency (<4 Hz) oscillatory dynamics that support memory and neural computation. In addition, functional magnetic resonance imaging (fMRI) studies measuring blood-oxygenation-level-dependent (BOLD) signals have demonstrated widespread hemodynamic alterations during NREM sleep. Sleep is also associated with increased interstitial fluid volume and clearance of metabolic waste products into the CSF, and clearance is stronger in sleep with more low-frequency EEG oscillations.” (Fultz N E et al. Coupled electrophysiological, hemodynamic, and cerebrospinal fluid oscillations in human sleep. Science. 2019:628-631.) (citations omitted).

Conventional treatment of AD and Parkinson's Disease by drugs, diet, physical activity, or mental training remains modestly successful. Current research suggests a new target for treatment for these and other neurodegenerative disorders affecting tens of millions of Americans. This target is the brain's glymphatic system, which moves cerebral fluid through the brain's perivascular spaces and interstitium, clearing waste products and toxins from the brain. Glymphatic functioning varies with the brain's neuronal activity, with sleep and wakefulness and with aging and with pathology. Glymphatic clearance is shown to be diminished in mouse models of AD, diminishing the clearance of β-amyloid and contributing to the accumulation of amyloid plaques, a prominent pathological feature of the brains of patients with AD.

Several experimental means of enhancing the glymphatic drainage for the brain have been suggested. One is the intermittent stimulation of neuronal activity to enhance glymphatic circulation. The coupling of neuronal activation and CSF dynamics may not be as strong in patients with neurodegenerative diseases as in young healthy subjects. Another means of enhancing glymphatic drainage may the use of pharmacologic agents to enhance AQP4 polarization that has a role in the glymphatic circulation.

B. The Implantable Medical Device

The previously described implantable medical device properly modified may create slow (<0.05 Hz) oscillations of intracranial pressure that have been shown in recent studies to enhance glymphatic flow (Fultz, Nina E., et al.). This implantable medical device is intended to provide a means for minimally invasive monitoring and modifying of intracranial pressures in patients with AD or other neurodegenerative disease. Specifically, the implantable medical device modulates cyclic CSF pressure oscillations related to the arterial pulse and the respiratory cycle transmitted to the cranial vault. A modification and improvement to the implantable medical device may modulate CSF pressure and circulation more flexibly.

The modified implantable medical device consists of a circular body which can be fasted to the skull. It has a hollow chamber with a rigid cap at one end and a membrane or disc or plate at the other end, depending on the intended use of the implantable medical device. The chamber may be accessed by removing the cap. The membrane or disc or plate may be accessed and replaced as needed.

The implantable medical device is implanted in the cranial vault at a site selected by the surgeon, likely in a region of the head that is easily accessible. A small craniotomy is placed in the skull; the implantable medical device is then inserted into in the craniotomy site and secured. The implanted medical device has rigid outer structure to protect against trauma to the brain and an inner part suited to the function of the device.

The implantable medical device includes functionality that allows access to its inner portion (chamber) either during its insertion into the skull or at a future date. This access includes: 1) a threaded opening in the rigid out-portion of the implantable medical device; 2) a threaded cap that can be screwed into the threaded opening to seal it; and 3) a tool to help the surgeon tighten the cap in the threaded opening. Such access allows the surgeon to exchange the mechanisms within the implantable medical device, either because of ordinary wear on the mechanisms or a need to change the properties of mechanisms.

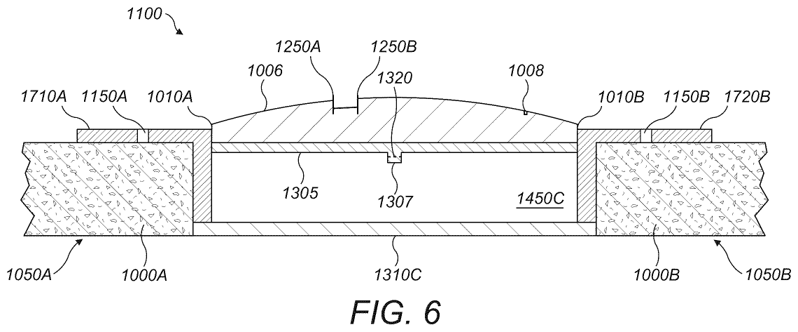

FIG. 6 shows a schematic 1100 of a modified implantable medical device shown in FIGS. 1-5. Here, the flanges 1150A, 1150B and the bonded areas 1710A, 1710B that attach to the calvarium 1000A, 1000B have been modified such that the chamber 1450C in the modified device may be accessed without removing the device from its position in the skull. The chamber 1450C has a flexible membrane 1310C at its inner end where it abuts the dural membrane of the calvarium's inner surface 1050A, 1050B. A circular hole with threads 1010A, 1010B is placed in the outer surface and a cap 1006 made of rigid material has been created to screw into the threaded hole 1010A, 1010B. The cap 1006 also has a slot 1008 that accepts a modified tool, like a screwdriver, which assists in the surgeons placing or removing the cap. The cap 1006 may be screwed to a variable degree to add pressure to the elastic structure within the chamber. The cap 1006 may incorporate one or more spacers 1250A, 1250B to increase the pressure on a device placed in the chamber, if needed.

The chamber 1450C may have a circular fitted disc, plate, or membrane 1305 to prevent fluids from entering the chamber. The circular fitted disc, plate, or membrane 1305 is selected depending on the mechanisms placed in the chamber 1450C. For mechanisms in the chamber 1450C that are elastic or designed for producing a pressure wave, a flexible membrane is selected. If a camera device is placed in the chamber 1450C, a transparent rigid plate may be used. For mechanisms in the chamber 1450C that inject into the subarachnoid space, a disc with an appropriately placed opening 1307 is used. The opening 1307 has a seal 1320 to prevent leakage around the conduit that leads to the subarachnoid space. For mechanisms in the chamber 1450C that monitor pressure in the subarachnoid space, the disc 1305 may instead have a flexible area through which pressure is transmitted.

The features shown in FIG. 6 are modifications of the implantable medical device shown in FIGS. 1-5. Such an implantable medical device may have any or all of the features shown in FIGS. 1-5. Further, this implantable medical device may have any or all of the additional or modified features shown in FIG. 6. Thus, the implantable medical devices may have any or all of the features shown in FIGS. 1-5, on the one hand, and FIG. 6, on the other hand.

C. Renewable Power for the Device

The implantable medical device may include a mechanism that can be placed in the chamber 1450C of the device with a source of renewable power that forces the implantable medical device to expand or collapse at a selected rate (such as in the range of 0.1 to 0.05 Hz) with a selectable volume change of up to 1 mL. The operation of the inserted mechanism increases intracranial pressure in the range of several pascals at a frequency imitating the cyclic intracranial pressure variations that drive the glymphatic flow in the brain during deep sleep. This feature of the implantable medical device allows the device to provide the type of pressure oscillations that characterize NREM sleep and that possibly drives the glymphatic CSF circulation in the brain. Recent studies demonstrate that this may remove toxic wastes from the brain. The glymphatic circulation possibly protects patients from neurodegenerative diseases, most notably AD. This also provides the opportunity to test in patients and in animals models of neuro degenerative disease where the efficacy of slow CSF oscillations during sleep to remove toxic wastes and restore or maintain neurologic functions may be determined.

Structures potentially useful to power the movement of the expanding-contracting feature for the implantable medical device include:
Motored mechanical motion.
Motorless mechanical motion.
Muscle Wire, which is an extremely thin wire made from Nitinol (a nickel-titanium alloy) that is known for its ability to contract when an electric current is applied.
Shape memory alloys such as Nitinol magnetic devices.
Piezo-electric materials such as lead zirconate titanate. This inorganic compound (with the chemical formula Pb[ZrₓTi₁₋ₓ]O₃ (0≤x≤1)), is a ceramic perovskite material that shows a marked piezoelectric effect, meaning that the compound changes shape when an electric field is applied. It is used in a number of practical applications such as ultrasonic transducers and piezoelectric resonators. Being piezoelectric, lead zirconate titanate physically changes shape when an external electric field is applied, which is useful for actuator applications. The relative permittivity of lead zirconate titanate ranges from 300 to 20,000 Farad/meter, depending upon orientation and doping.

Alternatively, the inner and outer surface of the appropriate portions of the implantable medical device may have electromagnetic parts so that an electric current causes them to attract or repel.

Power sources may include: rechargeable battery, replaceable battery, wireless transfer energy, ultrasonic energy harvesting, capacitive coupling link, and inductive energy harvesting.

For the implantable device applications, wireless transfer energy is currently considered to be a robust method to use instead of batteries. There may be three possible methods of wireless transfer energy:
1) Ultrasonic energy harvesting. “Ultrasonic transmission is a modern method of energy harvesting. This method is relatively safe for the human body and does not cause electronic interference with other electromagnetic devices. In 2004, Phillips et al. designed a device that allows pulsed ultrasound to provide a milliamp order of currents in piezoelectric devices. Tower et al. produced a device that may be suitable for potential monitoring. This device converts the energy of a surface-applied ultrasound beam to a high-frequency current. A receiver inside the device absorbs the ultrasound energy and converts it into electrical charge. The design is considerably higher than 100 pW of the 2-D electrostatic power harvester reported by Bartsch et al. In general, this method is under improvement to overcome disadvantages such as relatively low harvested energy and large size caused by MEMS devices.” Hannan, M. A et al. Energy harvesting for the implantable biomedical devices: issues and challenges. BioMed Eng OnLine 13:79 (2014) (citations omitted).
2) Capacitive coupling link. “The capacitive coupling link approach is used to transfer data and power in short wireless communications to the implanted devices. The basis for this approach is two parallel plates which behave like condensers. The first plate is attached to the skin outside of the body; the second plate is implanted inside the body and attached . . . to the implanted device.” Kanaan, A. I. et al. Implantable Wireless Systems: A Review of Potentials and Challenges. Antenna Systems. 4:4. 2021.
3) Inductive energy harvesting. This uses magnetic coupling as the communication environment, which is common with radio frequency identification techniques.

Additional information may be found in related references:
Massachusetts Institute of Technology. “Wireless system can power devices inside the body: New technology could enable remote control of drug delivery, sensing, and other medical applications.” ScienceDaily. ScienceDaily, 4 Jun. 2018. (“The implants are powered by radio frequency waves, which can safely pass through human tissues.”)
Singer & Robinson, Wireless Power Delivery Techniques for Miniature Implantable Bioelectronics, Advanced Healthcare Materials, Jun. 10, 2021. (“Progress in implanted bioelectronic technology offers the opportunity to develop more effective tools for personalized electronic medicine. While there are numerous clinical and pre-clinical applications for these devices, power delivery to these systems can be challenging. Wireless battery-free devices offer advantages such as a smaller and lighter device footprint and reduced failures and infections by eliminating lead wires. However, with the development of wireless technologies, there are fundamental tradeoffs between five essential factors: power, miniaturization, depth, alignment tolerance, and transmitter distance, while still allowing devices to work within safety limits. These tradeoffs mean that multiple forms of wireless power transfer are necessary for different devices to best meet the needs for a given biological target. Here six different types of wireless power transfer technologies used in bioelectronic implants—inductive coupling, radio frequency, mid-field, ultrasound, magnetoelectrics, and light—are reviewed in context of the five tradeoffs listed above. This core group of wireless power modalities is then used to suggest possible future bioelectronic technologies and their biological applications.”)
Khan S R, et al. Wireless Power Transfer Techniques for Implantable Medical Devices: A Review. Sensors (Basel). 2020 Jun. 19; 20(12):3487. (“Wireless power transfer (WPT) systems have become increasingly suitable solutions for the electrical powering of advanced multifunctional micro-electronic devices such as those found in current biomedical implants. The design and implementation of high power transfer efficiency WPT systems are, however, challenging. The size of the WPT system, the separation distance between the outside environment and location of the implanted medical device inside the body, the operating frequency and tissue safety due to power dissipation are key parameters to consider in the design of WPT systems. This article provides a systematic review of the wide range of WPT systems that have been investigated over the last two decades to improve overall system performance. The various strategies implemented to transfer wireless power in implantable medical devices (IMDs) were reviewed, which includes capacitive coupling, inductive coupling, magnetic resonance coupling and, more recently, acoustic and optical powering methods. The strengths and limitations of all these techniques are benchmarked against each other and particular emphasis is placed on comparing the implanted receiver size, the WPT distance, power transfer efficiency and tissue safety presented by the resulting systems. Necessary improvements and trends of each WPT techniques are also indicated per specific IMD.”)

D. Potential Benefits of the Device

Key features of the glymphatic circulation have been described, including changes in fluid dynamics resulting from periodic increases in cerebral blood volume during sleep. These changes result in an overall increase in CSF flow in the ventricular system and glymphatic circulation which increases the clearing of toxins and metabolites from the brain.

The circulatory changes during sleep are considered important to maintain brain health. These flow changes are not detected in patients with AD, depriving them of an important rinsing mechanism and increasing the risk of accumulating beta amyloid in the brain and reducing brain function. Hypothetically, treatment of AD with an implantable medical device that increases CSF circulation and increases CSF flow in the glymphatic system reduces or reverses the accumulation of the beta amyloid that is involved in the etiology of the disease. Based on current research a medical device that produces slow (<0.05 Hz) intracranial pressure changes would increase glymphatic flow.

One of the mechanisms that fits into the chamber may produce pressure waves in the cranial vault designed to promote glymphatic and/or CSF flow. Typically the waves are slow (0.05 Hz) and small in magnitude. Equipped with a wave generator, the mechanism transmits to the intracranial cerebrospinal fluid pressure oscillations similar to pressure waves associated with the periodic glymphatic flow in the brain during sleep.

While the placement of a pressure wave generator into the cranial vault is one use of the implantable medical device, other mechanisms may be placed that permit the injection of dyes or tracers or drugs or of the observing the surface of the brain. Sensors may be included in the implantable medical device to allow the monitoring of CSF pressure fluctuations and volume changes in the chamber.

With one or more mechanisms in the chamber, the implantable medical device may perform as a pressure wave damper, pressure wave generator, injector of tracers or dyes or drugs or chemotherapeutic agents, as a tool to measure CSF dynamics in experimental animals or patients injector to infuse drugs, or to visualize the brain surface directly or with ultrasound. The implantable medical device may be equipped with sensors to monitor CSF dynamics and devices to alter CSF dynamics, which represents a robust tool for neurosurgical management of a variety of conditions and for studying disease states in experimental animals.

With an elastic pad or elastic device placed in the chamber, this implantable medical device has applications in treating Idiopathic Syringomyelia or other neurologic conditions with abnormally large amplitude pressure fluctuations, such as normal pressure hydrocephalus or communicating hydrocephalus.

### VII. CONCLUSION

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. An implantable medical device, comprising:
a cover level having a removable cover top and a cover underside;
a membrane level, the membrane level comprising a circular tub-like structure having a plurality of membrane level securing members, a flexible membrane, a tapered side portion, and a chamber;
a retaining level, the retaining level comprising a ring-like structure having a substantially circular retaining member with a hollow center;
wherein the membrane level is attached to the cover underside by securing the plurality of membrane level securing members between the substantially circular retaining member and the cover underside so that the flexible membrane, the tapered side portion, and the chamber pass through the hollow center of the substantially circular retaining member;
wherein the chamber includes at least one mechanism;
wherein after the tapered side portion and the chamber are inserted in a skull of a patient, the flexible membrane abuts a dural membrane of the calvarium's inner surface; and
wherein the at least one mechanism selectively increases and decreases pressure in the chamber by ongoing displacement of the flexible membrane, thereby modulating cerebrospinal flow in the patient.

2. The implantable medical device as in claim 1, further comprising a plurality of skull-securing mechanism passing through the plurality of securing holes and into the skull of the patient.

3. The implantable medical device as in claim 2, wherein the removable cover top has at least one spacer.

4. The implantable medical device as in claim 1, wherein the modulating cerebrospinal flow in the patient occurs at 0.05 Hz.

5. The implantable medical device as in claim 1, wherein the at least one mechanism comprises a source of renewable power.

6. The implantable medical device as in claim 1, wherein the at least one mechanism comprises a piezo-electric material.

7. The implantable medical device as in claim 1, wherein volume of the chamber varies between 1 mL and 2 mL during a cardiac cycle of the patient.

8. The implantable medical device as in claim 1, further comprising a control sensor for managing a status of the at least one mechanism and the flexible membrane.

9. The implantable medical device as in claim 1 wherein the flexible membrane comprises bio-compatible ultra-high-molecular-weight polyethylene.

10. A cerebrospinal fluid flow diverter, comprising:
a cover level having a removable cover top side and a cover underside;
a membrane level comprising a plurality of membrane level securing members, a flexible membrane, a tapered side portion, and a chamber, wherein the flexible membrane forms the bottom boundary of the chamber;
a plurality of skull-securing mechanism secured through at least one of the cover level and the membrane level and into a skull of a patient;
wherein the chamber includes at least one mechanism;
wherein the at least one mechanism comprises a source of renewable power;
wherein after the tapered side portion and the chamber are inserted in a skull of a patient, the at least one mechanism selectively increases and decreases in pressure by the ongoing displacement of the flexible membrane, thereby modulating the cerebrospinal flow in the patient.

11. The implantable medical device as in claim 10, wherein the source of renewable power comprises at least one of rechargeable battery, replaceable battery, wireless transfer energy, ultrasonic energy harvesting, capacitive coupling link, and inductive energy harvesting

12. The implantable medical device as in claim 10, wherein the mechanism selectively increases and decreases in pressure occur as a result of muscle wire.

13. The implantable medical device as in claim 10, further comprising a membrane within the chamber that prevents fluids from entering the chamber.

14. The implantable medical device as in claim 10, further comprising a membrane within the chamber through which pressure is transmitted.

15. The implantable medical device as in claim 10, wherein the at least one mechanism comprises a camera.

16. The implantable medical device as in claim 10, wherein the diameter of the implantable medical device is between 11 mm and 18 mm.

17. The implantable medical device as in claim 10, wherein volume of the chamber varies between 1 mL and 2 mL during a cardiac cycle of the patient.

18. The implantable medical device as in claim 10, wherein the at least one mechanism is controlled via wireless communication protocols.

19. The implantable medical device as in claim 10, wherein the flexible membrane comprises bio-compatible ultra-high-molecular-weight polyethylene.

20. The implantable medical device as in claim 10, wherein the removable cover top side comprises a slot for assistance in removing the removable cover top side.
